(19) United States
(12) Patent Application Publication
(10) Pub. No.: US 2016/0249825 A1
(43) Pub. Date: Sep. 1, 2016

(54) APPARATUS AND METHOD FOR CONCENTRATING MAGNETIC FIELD AT HIGH RESOLUTION AND MAGNETIC FIELD RECEIVING DEVICE FOR SAME

(71) Applicant: KOREA ADVANCED INSTITUTE OF SCIENCE AND TECHNOLOGY, Yuseong-gu Daejeon (KR)

(72) Inventors: Chun-taek RIM, Daejeon (KR); Eun-Soo LEE, Daejeon (KR); Junpil CHEON, Daejeon (KR); Bo-hwan CHOI, Anyang-si (KR)

(73) Assignee: KOREA ADVANCED INSTITUTE OF SCIENCE AND TECHNOLOGY, Daejeon (KR)

(21) Appl. No.: 15/029,085

(22) PCT Filed: Oct. 21, 2014
(86) PCT No.: PCT/KR2014/009901

§ 371 Date: Apr. 13, 2016

(30) Foreign Application Priority Data
Oct. 24, 2013 (KR) 10-2013-0127402

(51) Int. Cl. A61B 5/05; G01V 3/10; A61N 2/02

(52) U.S. Cl. CPC A61B 5/0522; A61N 2/02; G01V 3/104

(57) ABSTRACT

Embodiments of the present disclosure provide a magnetic-field focusing apparatus including a magnetic-field generating unit including a plurality of transmit coils and configured to receive a transmit pulse to be applied to each of the transmit coils and to generate a magnetic field, and a pulse generating unit configured to calculate a magnitude of a current to be applied to each of the transmit coils by using structure information based on a positional relationship between a focusing point for focusing a magnetic flux and the transmit coils and to generate the transmit pulse to be applied to each of the transmit coils based on the magnitude of the current. A magnetic-field receiving apparatus is also provided for the magnetic-field focusing apparatus.

### TECHNICAL FIELD

The present disclosure relates to a method and an apparatus for focusing a magnetic field at high resolution and an apparatus for receiving a magnetic field.

### BACKGROUND

The statements in this section merely provide background information related to some embodiments of the present disclosure and do not necessarily constitute prior art.

A magnetic induction tomography (MIT) applies a high-frequency magnetic field to a specific site of an object to be observed by using a plurality of transmit coils of a length of several centimeters, and detects a fine magnetic field generated by an eddy current magnetically induced inside the object by using a receive coil.

FIG. 1 is a phasor diagram of a weak magnetic field (ΔB) generated by an eddy current when a strong main magnetic field (B) is applied.

However, when transmit and receive coils are used as described above, a mutual attenuation of the magnetic field due to mutual interference therebetween and the high frequency leads to low resolution and poor SNR (Signal-to-Noise Ratio), which is insufficient for application to the human body for medical purposes.

### DISCLOSURE

### Technical Problem

The present disclosure has been achieved in view of the above aspect, and it is an object of some embodiments of the present disclosure to provide a method and an apparatus for focusing a magnetic field in an arbitrary position with high precision by using a high-precision coil and a high integrated sensor array.

### SUMMARY

In order to achieve the above-mentioned object, according to some embodiments of the present disclosure, an apparatus for focusing a magnetic field includes a magnetic-field generating unit including a plurality of transmit coils and configured to receive a transmit pulse to be applied to each of the transmit coils and to generate a magnetic field, and a pulse generating unit configured to calculate a magnitude of a current to be applied to each of the transmit coils by using structure information based on a positional relationship between a focusing point for focusing a magnetic flux and the plurality of transmit coils and to generate the transmit pulse to be applied to each of the plurality of transmit coils based on the calculated magnitude of the current.

According to some embodiments, the apparatus may further include a receive sensor unit including a plurality of receive coils each configured to generate an induction signal by a magnetic field focused on the focusing point and a measuring unit configured to measure a magnitude of the induction signal generated by each of the receive coils.

According to some embodiments, the measuring unit may be configured to measure the induction signal at a predetermined time after the transmit pulse is generated. The transmit pulse may include a positive-voltage pulse and a negative-voltage pulse.

According to some embodiments, the pulse generating unit may include a focusing-point calculating unit, a structure-information calculating unit and a circuit driving unit. The focusing-point calculating unit is configured to calculate information on the focusing point. The structure-information calculating unit is configured to calculate structure informations based on positional relationships of a plurality of focusing points with respect to a plurality of transmit coils. The circuit driving unit is configured to generate the transmit pulse based on the information on the focusing point and the structure information. The focusing-point calculating unit may be configured to receive an information on one or more focusing points which are candidates for focusing point setting.

In order to achieve the above-mentioned object, some embodiments of the present invention provide a method, performed by a magnetic flux focusing apparatus including a plurality of transmit coils, for focusing a magnetic field at high resolution. The method includes acquiring information on a focusing point for focusing the magnetic field, acquiring structure information based on positional relationships between the transmit coils and the focusing point, calculating a magnitude of a current to be generated for each of the plurality of transmit coils based on the structure information, and generating a transmission pulse for each of the plurality of transmit coils based on the magnitude of the current.

According to some embodiments, an apparatus for receiving a magnetic field includes a Hall element having a rod shape, and an electrode array including externally at least one of a plurality of first pairs of electrodes or a plurality of second pairs of electrodes. The first pairs of electrodes are diametrically opposite each other with respect to the Hall element with reference to a first direction. The second pairs of electrodes are diametrically opposite each other with respect to the Hall element with reference to a second direction. The first direction is perpendicular to the second direction and a longitudinal direction of the Hall element.

According to some embodiments, the Hall element is divided into a plurality of cells in the longitudinal direction, and each of the cells includes at least one of a first pair of electrodes or a second pair of electrodes.

According to some embodiments, an electrode constituting the first pair of electrode and the second pair of electrode may include a linear-shaped electrical conductor. The Hall element may be shaped to define a zigzag pattern of current flowing across the Hall element.

According to some embodiments, the apparatus for receiving the magnetic field may further include multiples of the Hall element in an array so that adjacent Hall elements have currents which are directed opposite to each other. With the Hall element shaped to define a zigzag pattern of current flowing across the Hall element, the apparatus may include two of the Hall element configured so that two adjacent Hall elements are arranged to face each other and have terminals interconnected on one side.

### Advantageous Effects

As described above, according to some embodiments of the present disclosure, the resolution of focusing the magnetic field can be dramatically improved by increasing the number of transmissions and receptions of magnetic field signals by removing an interference between adjacent coils for generating magnetic fields and temporally separating signals between the transmit coils and the receive coils by using a pulsed magnetic field.

In addition, using this technology enables an image to be obtained through a wall, and hence it can be used for military/counterterror applications, providing a source technology that enables a metal detection and an underground search.

By leveraging such fundamental technology, innovative developments are offered for general industrial purposes such as a novel cancer treatment, destroying cancer cells by irradiating a strong magnetic flux on a site of a human body, a long-range wireless charging, 3-dimensional magnetic flux communications and plasma magnetic flux pattern control for nuclear fusion.

### BRIEF DESCRIPTION OF DRAWINGS

FIG. 1 is a phasor diagram of a case of applying a strong principal magnetic flux B, wherein a minute magnetic flux ΔB is generated by an eddy current.

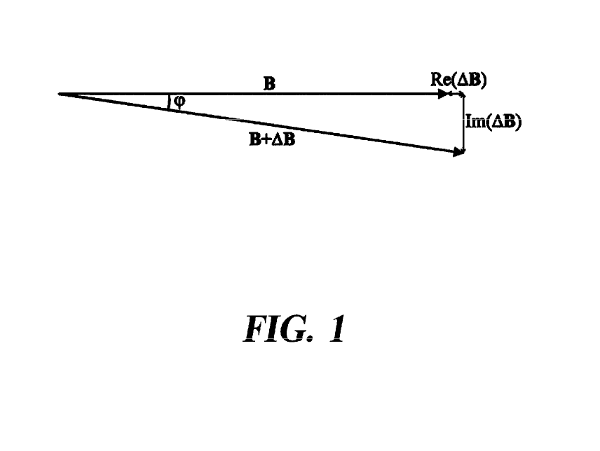

FIG. 2 is a diagram of a high-resolution magnetic-field focusing apparatus 100 according to some embodiments of the present disclosure.

FIG. 3 is a diagram of a shape of a magnetic-field generating unit 120.

FIG. 4 is a diagram for describing the intensity (or strength) of a magnetic flux generated in a particular position by a plurality of current sources which are arranged one-dimensionally.

FIG. 5 is a diagram for describing the intensity (or strength) of a magnetic flux generated in a particular position by a plurality of current sources which are arranged three-dimensionally.

FIG. 6 is a diagram of a structure of a pulse generating unit 110.

FIG. 7 is a diagram of a circuit driving unit 630 inclusive of a transmit coil 121.

FIG. 8 is an equivalent circuit diagram of the circuit driving unit 630, a human body 810 on which the magnetic flux is focused, a receive sensor unit 130 and a measuring unit 140.

FIG. 9 is a diagram of waveforms of voltage v_{S} and current i_{S} which are provided to the transmit coil 121.

FIG. 10 is a graph showing a current generated in the human body 810 when the magnetic flux is transferred to the human body 810 by the transmit coil 121.

FIG. 11 is a diagram of an induced voltage detected by a receive coil 131.

FIG. 12 is a flowchart of a method for high-resolution magnetic flux focusing according to some embodiments of the present disclosure.

FIG. 13 is a perspective view of a magnetic-field receiving apparatus 1300 according to some embodiments of the present disclosure.

FIG. 14 is a cross-sectional view of a first cell in FIG. 13 taken along line A-A′ and viewed in direction X.

FIG. 15 is a diagram of the shape of an electrode that can be used in the magnetic-field receiving apparatus 1300.

FIG. 16 is a diagram of a Hall element having two of the Hall element 1310 shown in FIG. 13 with one terminally protruding perpendicular from the other.

FIG. 17 is a diagram of the magnetic-field receiving apparatus 1300 with an optional zigzag configuration of the Hall element.

FIG. 18 is a diagram of a method for arranging a plurality of Hall elements.

FIG. 19 is a diagram showing another example of the shape of the Hall element.

REFERENCE NUMERALS

| 100 | High-resolution magnetic-field focusing apparatus | | |
|---|---|---|---|
| 110 | Pulse generating unit | 120 | Magnetic-field generating unit |
| 121 | Transmit coil | 130 | Receive sensor unit |
| 131 | Receive coil | 140 | Measuring unit |
| 610 | Focusing-point calculating unit | | |
| 620 | Structure-information calculating unit | | |
| 630 | Circuit driving unit | 710 | Control unit |
| 720 | DA converter | 730 | Operational amplifier |
| 740 | Gate driver | 1300 | Magnetic-field receiving apparatus |

1310, 1610, 1710, 1810, 1820, 1830, 1910, 1920 Hall element

| | | | |
|---|---|---|---|
| 1311 | First cell | 1312 | Second cell |
| 1313 | N-th cell | | |

| | |
|---|---|
| 1321, 1322, 1621, 1622 | First pair of electrodes |
| 1323, 1324, 1623, 1624 | Second pair of electrodes |

| | | | |
|---|---|---|---|
| 1510 | Connector | 1611 | Protrusion |
| 1910 | First Hall element | 1911 | One terminal of first Hall element |

| | |
|---|---|
| 1920 | Second Hall element |
| 1921 | One terminal of second Hall element |

### DETAILED DESCRIPTION

Hereinafter, at least one embodiment of the present disclosure will be described in detail with reference to the accompanying drawings. In the following description, like reference numerals designate like elements, although the elements are shown in different drawings. Further, in the following description of the at least one embodiment, a detailed description of known functions and configurations incorporated herein will be omitted for the purpose of clarity and for brevity.

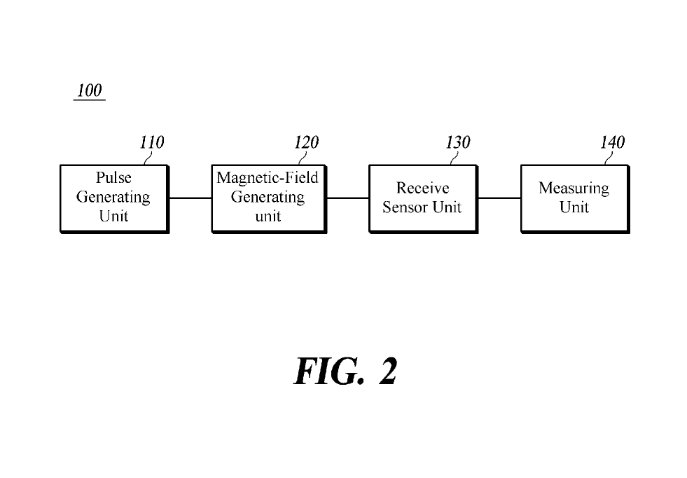

FIG. 2 is a diagram of a high-resolution magnetic-field focusing apparatus 100 according to some embodiments of the present disclosure.

As shown in FIG. 2, the high resolution magnetic-field focusing apparatus 100 according to some embodiments of the present disclosure includes a pulse generating unit 110, a magnetic-field generating unit 120, a receive sensor unit 130 and a measuring unit 140, although some components may be added to or dropped from those specified above depending on the embodiment of the present disclosure in implementing the high resolution magnetic-field focusing apparatus 100.

The magnetic-field generating unit 120 may include a plurality of transmit coils arranged horizontally and vertically on a plane. Here, the arrangement of the transmit coils is described to be planar, but not limited thereto, and a variety of arrangements are possible depending on the embodiment, such as an array on a curved surface or a stereoscopic or three-dimensional arrangement.

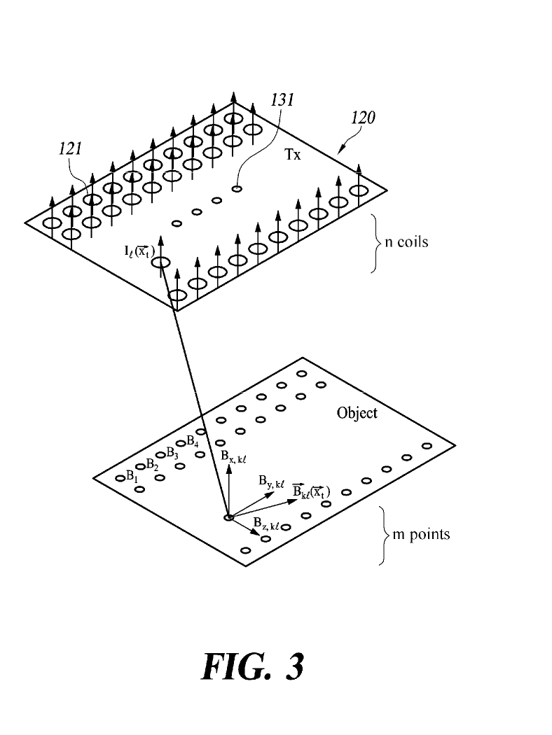

FIG. 3 is a diagram illustrating a shape of the magnetic-field generating unit 120. The magnetic-field generating unit 120 shown in FIG. 3 includes a plurality of transmit coils 121, arranged horizontal and vertical. The transmit coils 121 can also be arranged in a three-dimensional form instead of the arrangement on a two-dimensional plane. In this case, the transmit coils 121 can be arranged in a horizontal direction, a vertical direction and a height direction. Other various forms can be adopted in arranging the transmit coils 121. A receive coil 131, which will be described later, can be arranged adjacent to the transmit coils 121.

The pulse generating unit 110 calculates the magnitude of a current to apply to each of the plurality of transmit coils 121 by using the structure information based on the positional relationship between the desired magnetic flux focusing point and the transmit coils 121, and generates and applies a transmit pulse to each of the transmit coils 121 based on the calculated magnitude of the current.

The pulse generating unit 110 can generate pulses having a predetermined magnitude simultaneously for the transmit coils 121, and can generate a pulse having a predetermined magnitude independently for every one of the transmit coils 121.

In some embodiments, when the pulse generating unit 110 generates a pulse of an appropriate magnitude for application to the plurality of transmit coils 121, the magnetic flux is focused on a specific point.

In some embodiments, an SMF (High-Resolution Synthesized Magnetic flux Focusing) technique is proposed, which utilizes a plurality of (e.g., several thousands) transmit coils that generates precisely controlled pulse currents for focusing a magnetic field on a specific point.

Some embodiments of the present disclosure enable magnetic fields generated from a plurality of transmitting coils distant from a human body or an object by a predetermined distance to be synthesized and to be focused only on a specific point in the human body or object.

Further, the generation of a transmit pulse from the transmit coil is temporally separated from the reception of a pulse signal by the receive coil to detect a residual current induced in the human body or the object.

Moreover, in some embodiments, not only the receive sensor unit can synthesize magnetic fields, but also a circuit constituting the magnetic-field generating unit can synthesize the magnetic fields, which cannot be found in any previous papers or patents. Also provided are equations for calculating the intensity of a synthesized magnetic field by a linear matrix.

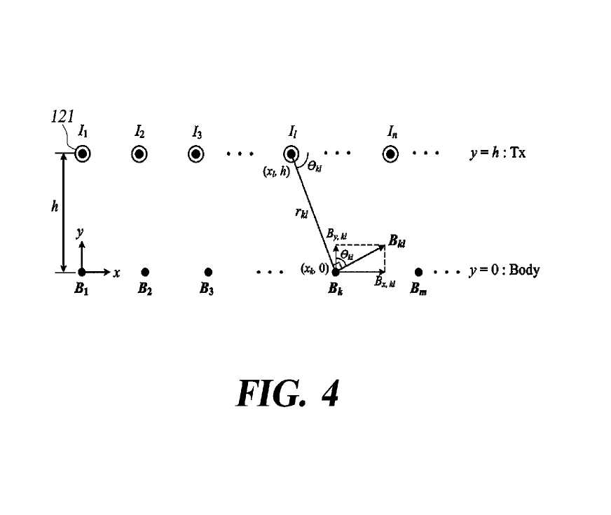

FIG. 4 is a diagram for describing the intensity (or strength) of a magnetic flux generated in a particular position by a plurality of current sources which are arranged one-dimensionally.

In FIG. 4, a magnetic flux density \vec{Bₖₗ} generated at a specific point (xₖ, 0) by a current source I_{L} among current sources I₁, I₂, . . . , and Iₙ is defined by Equation 1.

$$\vec{{B}_{kl}} = {B}_{x , kl} \vec{{x}_{0}} + {B}_{y , kl} \vec{{y}_{0}} = \frac{{μ}_{0} {I}_{l}}{2 π {r}_{kl}} ( sin {θ}_{kl} \vec{{x}_{0}} + cos {θ}_{kl} \vec{{y}_{0}} ) \\ ∵ {r}_{k , l} = \sqrt{{( {x}_{k} - {x}_{l} )}^{2} + {h}^{2}} , \\ {θ}_{kl} = {tan}^{- 1} ( \frac{y}{{x}_{k} - {x}_{l}} ) \qquad Equation 1$$

Therefore, a sum \vec{Bₖ} of magnetic flux densities generated at a specific point (xₖ, 0) by all current sources I₁, I₂, . . . , and Iₙ is defined by Equation 2.

$$\vec{{B}_{k}} = {\sum }_{l = 1}^{n} \vec{{B}_{kl}} = \vec{{x}_{0}} {\sum }_{l = 1}^{n} {B}_{x , kl} + \vec{{y}_{0}} {\sum }_{l = 1}^{n} {B}_{y , kl} ≡ {B}_{x , k} \vec{{x}_{0}} + {B}_{y , k} \vec{{y}_{0}} \qquad Equation 2$$

Two components B_{x,k} and B_{y,k} of the intensity of the magnetic field at a plurality of points k (k=1, . . . , m) in Equation 2 are respectively represented as matrices in Equation 3.

$$[ \begin{matrix}{B}_{x , 1} \\ {B}_{x , 2} \\ ⋮ \\ {B}_{x , m}\end{matrix} ] = [ \begin{matrix}{a}_{11 x} & {a}_{12 x} & … & {a}_{1 nx} \\ {a}_{21 x} & {a}_{22 x} & … & {a}_{2 nx} \\ ⋮ & ⋮ & & ⋮ \\ {a}_{m 1 x} & {a}_{m 2 x} & … & {a}_{mnx}\end{matrix} ] [ \begin{matrix}{I}_{1} \\ {I}_{2} \\ ⋮ \\ {I}_{n}\end{matrix} ] ⇔ {B}_{x} = {A}_{x} I , \\ ∵ {a}_{klx} = \frac{{μ}_{0}}{2 π {r}_{kl}} sin {θ}_{kl} \\ [ \begin{matrix}{B}_{y , 1} \\ {B}_{y , 2} \\ ⋮ \\ {B}_{y , m}\end{matrix} ] = [ \begin{matrix}{a}_{11 y} & {a}_{12 y} & … & {a}_{1 ny} \\ {a}_{21 x} & {a}_{22 x} & … & {a}_{2 ny} \\ ⋮ & ⋮ & & ⋮ \\ {a}_{m 1 x} & {a}_{m 2 x} & … & {a}_{mny}\end{matrix} ] [ \begin{matrix}{I}_{1} \\ {I}_{2} \\ ⋮ \\ {I}_{n}\end{matrix} ] ⇔ {B}_{y} = {A}_{y} I , \\ ∵ {a}_{kly} = \frac{{μ}_{0}}{2 π {r}_{kl}} cos {θ}_{kl} \qquad Equation 3$$

As shown in Equation 3, each of the values aₖₗₓ and a_{kly} is determined based on the geometric positional relationship between the specific points of the given current sources I₁, I₂, . . . , and Iₙ, and hence the x and y components of the magnetic flux densities at a plurality of specific points can be obtained for the current sources I₁, I₂, . . . , and Iₙ.

Equation 3 can be modified to be Equation 4.

$$B ≡ [ \begin{matrix}{B}_{x} \\ {B}_{y}\end{matrix} ] , \\ A ≡ [ \begin{matrix}{A}_{x} \\ {A}_{y}\end{matrix} ] B = AI \qquad Equation 4$$

When n=2m in Equation 4, Equation 5 is obtained, where n is the number of transmit coils 121, and m is the number of points on the reception side where the magnetic flux is focused.

I=A⁻¹B Equation 5

Accordingly, as shown in Equation 5, the magnitude of the current flowing to each of the given current sources I₁, I₂, . . . , and Iₙ can be calculated to obtain the magnetic flux density of a desired magnitude at the plurality of specific points based on the positional relationship with the current sources I₁, I₂, . . . , and Iₙ.

Referring to FIG. 3, a description will be given with respect to the intensity (or strength) of the magnetic field generated at a specific point by a plurality of two-dimensionally arrayed current sources.

In FIG. 3, a sum of the magnetic flux density generated at a specific point \vec{x_{b}} by all the current sources I₁, I₂, . . . , and Iₙ is defined by Equation 6.

$$\vec{{B}_{k}} ( \vec{{x}_{b}} ) = {\sum }_{l = 1}^{n} = {B}_{kl} = {\sum }_{l = 1}^{n} {I}_{i} ( \vec{{x}_{0}} {a}_{klx} + \vec{{y}_{0}} {a}_{kly} + \vec{{z}_{0}} {a}_{klz} ) \\ ∵ {a}_{klk} = {f}_{klx} ( \vec{{x}_{b}} - \vec{{x}_{t}} ) , \\ {a}_{kly} = {f}_{kly} ( \vec{{x}_{b}} - \vec{{x}_{t}} ) , \\ {a}_{klz} = {f}_{klz} ( \vec{{x}_{b}} - \vec{{x}_{t}} ) \qquad Equation 6$$

Three components of the magnetic flux density at the specific point \vec{x_{b}} in Equation 6 is defined as Equation 7.

$${B}_{x , k} = {\sum }_{l = 1}^{n} {a}_{kIk} {I}_{l} , \\ {B}_{y , k} = {\sum }_{l = 1}^{n} {a}_{kIy} {I}_{l} , \\ {B}_{z , k} = {\sum }_{l = 1}^{n} {a}_{zkI} {I}_{l} \qquad Equation 7$$

Example x, y, z components B_{x,k}, B_{y,k} and B_{z,k} of the intensity of the magnetic field at the plurality of points k in Equation 7 are respectively represented as matrices in Equation 8.

$${{B}_{x}}_{( m \times 1 )} = [ \begin{matrix}{B}_{x , 1} \\ {B}_{x , 2} \\ ⋮ \\ {B}_{x , m}\end{matrix} ] = [ \begin{matrix}{a}_{11 x} & {a}_{12 x} & … & {a}_{1 nx} \\ {a}_{21 x} & {a}_{22 x} & … & {a}_{2 nx} \\ ⋮ & ⋮ & & ⋮ \\ {a}_{m 1 x} & {a}_{m 2 x} & … & {a}_{mnx}\end{matrix} ] [ \begin{matrix}{I}_{1} \\ {I}_{2} \\ ⋮ \\ {I}_{n}\end{matrix} ] ⇔ {{A}_{x}}_{( m \times n )} {I}_{( n \times 1 )} \\ {{B}_{y}}_{( m \times 1 )} = [ \begin{matrix}{B}_{y , 1} \\ {B}_{y , 2} \\ ⋮ \\ {B}_{y , m}\end{matrix} ] = [ \begin{matrix}{a}_{11 y} & {a}_{12 y} & … & {a}_{1 ny} \\ {a}_{21 y} & {a}_{22 y} & … & {a}_{2 ny} \\ ⋮ & ⋮ & & ⋮ \\ {a}_{m 1 y} & {a}_{m 2 y} & … & {a}_{mny}\end{matrix} ] [ \begin{matrix}{I}_{1} \\ {I}_{2} \\ ⋮ \\ {I}_{n}\end{matrix} ] ⇔ {{A}_{y}}_{( m \times n )} {I}_{( n \times 1 )} \\ {{B}_{z}}_{( m \times 1 )} = [ \begin{matrix}{B}_{z , 1} \\ {B}_{z , 2} \\ ⋮ \\ {B}_{z , m}\end{matrix} ] = [ \begin{matrix}{a}_{11 z} & {a}_{12 z} & … & {a}_{1 nz} \\ {a}_{21 z} & {a}_{22 z} & … & {a}_{2 nz} \\ ⋮ & ⋮ & & ⋮ \\ {a}_{m 1 z} & {a}_{m 2 z} & … & {a}_{mnz}\end{matrix} ] [ \begin{matrix}{I}_{1} \\ {I}_{2} \\ ⋮ \\ {I}_{n}\end{matrix} ] ⇔ {{A}_{z}}_{( m \times n )} {I}_{( n \times 1 )} \qquad [ Equation 8 ]$$

As defined by Equation 8, aₖₗₓ, a_{kly} and a_{klz} are determined based on the geometric positional correlation of the specific point \vec{x_{b}} with respect to the given current sources I₁, I₂, . . . , and Iₙ, and hence the x, y, and z components of the magnetic flux at the plurality of specific points can be obtained with respect to the current sources I₁, I₂, . . . , and Iₙ.

Modifying Equation 8 can obtain Equation 9.

$$B {=}^{Δ} [ \begin{matrix}{B}_{x} \\ {B}_{y} \\ {B}_{z}\end{matrix} ] , \\ A {=}^{Δ} [ \begin{matrix}{A}_{x} \\ {A}_{y} \\ {A}_{z}\end{matrix} ] I \to B = AI \\ ( 3 m \times 1 ) ( 3 m \times n ) \qquad Equation 9$$

If n=3m, as in the case of Equation 4, Equation 9 can be represented as Equation 5, where B can be represented by a 3m×1 matrix, and A can be represented by a 3m×n matrix, n being the number of transmit coils 121, and m being the number of points on the reception side where the magnetic flux is focused.

Accordingly, as shown in Equation 5, the magnetic-field generating unit 120 in the configuration of FIG. 3 is capable of obtaining the magnitude of the current flowing through the current sources I₁, I₂, . . . , and Iₙ to obtain the magnetic flux of the desired intensity at the specific points based on the positional correlation of the current sources I₁, I₂, . . . , and Iₙ.

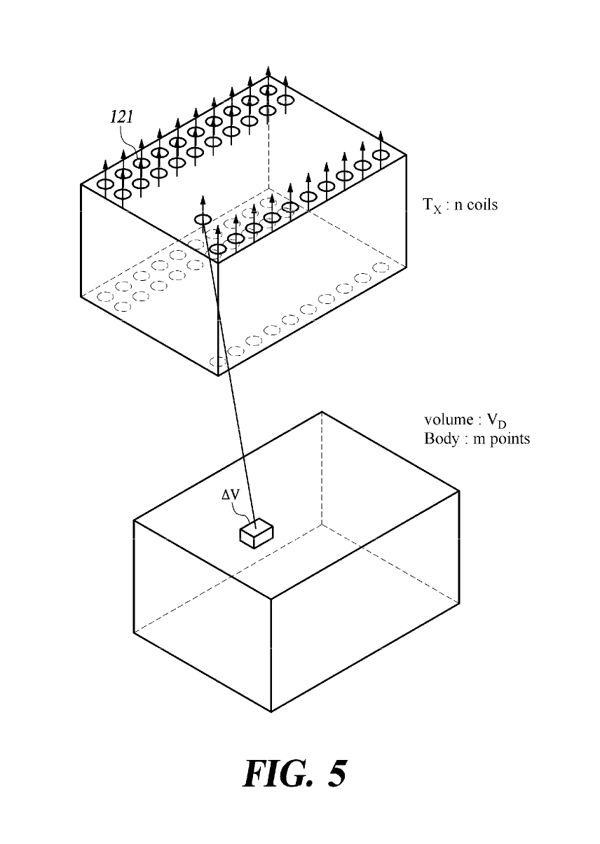

FIG. 5 is a diagram for describing the intensity (or strength) of a magnetic flux generated in a particular position by a plurality of current sources which are arranged three-dimensionally.

The intensity of the magnetic field generated at a specific point by a plurality of current sources including a plurality three-dimensionally arranged of transmit coils 121 can now be obtained as in the case of the two-dimensional array:

$$B = [ \begin{matrix}{B}_{x} \\ {B}_{y} \\ {B}_{z}\end{matrix} ] = [ \begin{matrix}{A}_{x} \\ {A}_{y} \\ {A}_{z}\end{matrix} ] I \to B = AI \\ ( 3 m \times 1 ) ( 3 m \times n ) ( n \times 1 ) \qquad Equation 10$$

As with the two-dimensional array, the three-dimensional array of the transmit coils 121 provides Equation 10 which, if n=3m, can be represented as Equation 5. Here, B may be represented by a 3m×1 matrix, A by a 3m×n matrix, and I by an n×1 matrix, n being the number of transmit coils 121, and m being the number of points on the reception side where the magnetic flux is focused.

In FIG. 5, the volume resolution ΔV of a receiving end (which means a human body that receives a magnetic flux) of the magnetic flux at the transmit coil 121 is a value obtained by dividing the entire reception end volume V_{b} by the number of points on the reception side as Equation 11.

$$Δ V = \frac{{V}_{b}}{m} \geq \frac{{V}_{b}}{n / 3} = \frac{3 {V}_{b}}{n} \qquad Equation 11$$

When expressing Equation 5 in normalized determinant, it can be represented as Equation 12.

I_{N}=A⁻¹B_{N} [symbol] CB_{N} ∵ C [symbol] A⁻¹ Equation 12

When it is desired to focus the magnetic flux generated by the transmit coil 121 at any one point on the reception side, 1 is put in the intensity of the magnetic field for just one element (k-th component) and 0s in the intensities of the magnetic field for all the remaining components in Equation 12, which provides the magnitude of the current required for the transmit coils 121 as Equation 13.

$$I = [ \begin{matrix}{C}_{11} & {C}_{12} & … & {C}_{1 n} \\ {C}_{21} & {C}_{22} & … & {C}_{2 n} \\ & ⋮ & & \\ {C}_{n 1} & {C}_{n 2} & … & {C}_{m}\end{matrix} ] [ \begin{matrix}0 \\ ⋮ \\ 1 \\ ⋮ \\ 0\end{matrix} ] \qquad Equation 13$$

As described above, the value of the matrix C in Equation 13 can be determined from the positional relationship between the transmit coils 121 and the plurality of points on the reception side.

The pulse generating unit 110 generates pulses having respectively predetermined magnitudes of electric current at the respective transmit coils 121 at the same time, and the predetermined magnitudes of electric current can be determined based on Equation 13. The pulse generating unit 110 stores the value of the matrix C in Equation 13 to generate the current of the predetermined magnitude. Further, the pulse generating unit 110 stores information on one or more points where the magnetic flux is focused. Accordingly, when a user inputs a position to be focused with the magnetic flux and the intensity of the magnetic field, the pulse generating unit 110 receives the inputs and drives the driving circuit of the transmit coils 121, thus generating the required current.

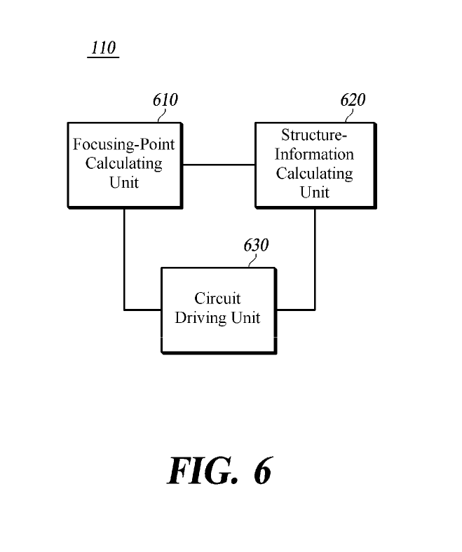

FIG. 6 is a diagram of a structure of a pulse generating unit 110.

As shown in FIG. 6, the pulse generating unit 110 includes a focusing-point calculating unit 610, a structure-information calculating unit 620 and a circuit driving unit 630.

The focusing-point calculating unit 610 calculates the information on the desired focusing point to focus the magnetic flux. The information on the desired focusing point can be inputted by the user from a plurality of focusing points which are candidates for focusing point setting, and the inputted information on the focusing point includes one or more focusing points.

The structure-information calculating unit 620 calculates the structure information according to the positional relationship with transmit coils 121 and characteristics of a magnetic-flux transfer medium for each of the candidate focusing points. Such structure information is calculated for each of the candidates focusing points and stored in a memory in advance.

The circuit driving unit 630 generates pulses for generating currents of magnitudes calculated by Equation 12 or 13 at all of the transmit coils 121 independently, based on the information on the desired focusing point of the magnetic flux and the information calculated by the structure-information calculating unit 620.

In some embodiments, the high-resolution magnetic-field focusing apparatus 100 further includes the receive sensor unit 130 and the measuring unit 140.

The magnetic flux generated by the current generated by the magnetic-field generating unit 120 can be focused on a point from the candidate focusing points, or can be sent to several points among the candidate focusing points.

The receive sensor unit 130 includes at least one receive coil 131 for generating an induction signal by a magnetic field focused on the focusing point. That is, the receive sensor unit 130 includes at least one receive coil 131 for generating the induction signal by a linkage of the magnetic flux transmitted by a transmit pulse generated at the plurality of transmit coils 121 of the magnetic-field generating unit 120. The plurality of receive coils 131 can be arranged close to the receive coil 131 as shown in FIG. 3; however, the position of the receive coil 131 is not limited to this, but can be arranged at various locations depending on the application purpose.

The measuring unit 140 measures the magnitude of the induced voltage generated as the induction signal generated by each of the at least one receive coil 131.

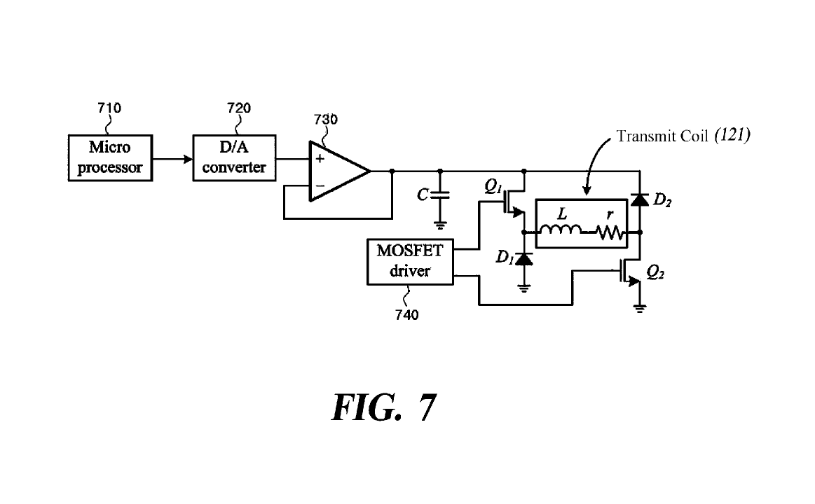

FIG. 7 is a diagram of the circuit driving unit 630 inclusive of a transmit coil 121.

In FIG. 7, portions excluding the transmit coil 121 are included in the circuit driving unit 630.

As shown in FIG. 7, the circuit driving unit 630 includes a control unit 710, a DA converter 720, an operational amplifier 730, a gate driver 740, a capacitor C, a first diode D1, a second diode D2, a first switch Q1 and a second switch Q2.

The circuit driving unit 630 includes the control unit 710 for the whole apparatus, and includes the DA converter 720, the operational amplifier 730, the gate driver 740, the capacitor C, the first diode D1, the second diode D2, the first switch Q1 and the second switch Q2 for each of the transmit coils 121.

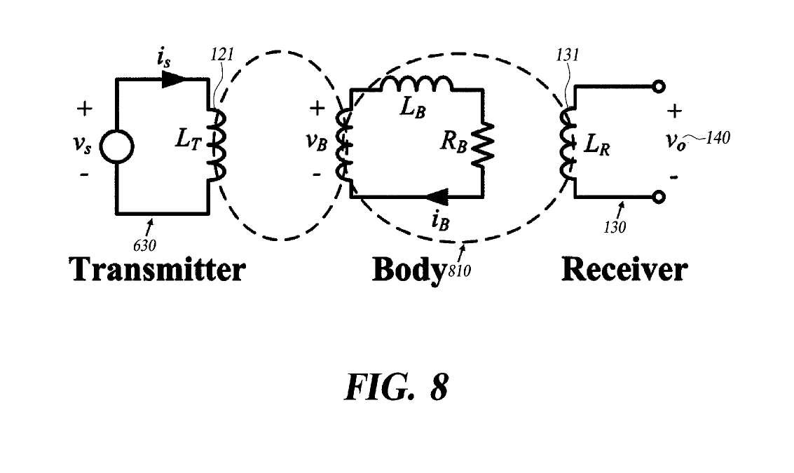

FIG. 8 is an equivalent circuit diagram of the circuit driving unit 630, a human body 810 on which the magnetic flux is focused, the receive sensor unit 130 and the measuring unit 140. Although some embodiments describe the human body 810 as an example, the object to which the magnetic flux is transferred is not limited to the human body, but can be various objects including objects in the ground, buildings, underwater.

In FIG. 7, when the control unit 710 sets the information on a voltage to be applied to the DA converter 720, the DA converter 720 converts the voltage into an analog value and provides it to the operational amplifier 730. In this manner, the control unit 710 sets voltages for all the transmit coils 121.

The control unit 710 drives the gate driver 740 to turn on the first switch Q1 and the second switch Q2 to supply the current to the transmit coil 121. In a predetermined time of supplying the current to the transmit coil 121, the control unit 710 turns off the first switch Q1 and the second switch Q2 to cut off the current supplied to the transmit coil 121.

When the current supplied to the transmit coil 121 is cut off, a parasitic ringing is generated by the first diode D1 and the second diode D2, which is gradually attenuated to zero current i_{S}.

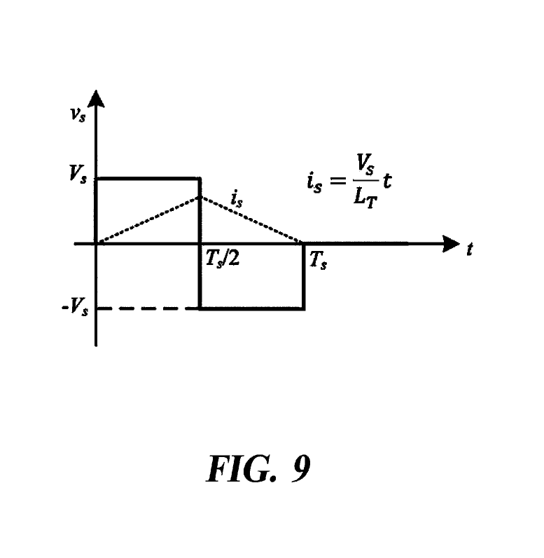

FIG. 9 is a diagram of waveforms of voltage v_{S} and current i_{S} which are provided to the transmit coil 121.

As shown in FIG. 9, when +V_{S} is supplied as a positive voltage pulse, current iₛ starts to increase, and when −V_{S} is supplied as a negative voltage pulse at a time T_{S}/2, current i_{S} starts to decrease. The supply of the negative voltage pulse is stopped at time T_{S}, and at this moment, current i_{S} becomes zero. While current i_{S} increases, the magnitude of i_{S} becomes i_{S}=V_{S}*T/L_{T}, where L_{T} is inductance of the transmit coil 121.

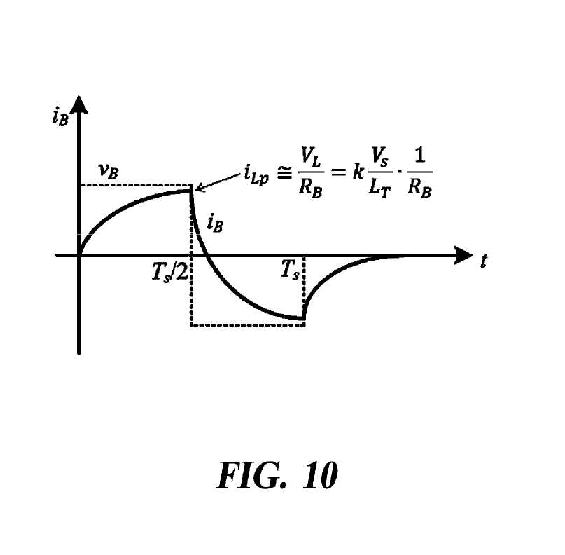

FIG. 10 is a graph showing a current generated in the human body 810 when the magnetic flux is transferred to the human body 810 by the transmit coil 121.

Referring to FIGS. 8 to 10, at the start of applying current i_{S} to the transmit coil 121, an induction current i_{LP} is generated in the human body 810. FIG. 10 shows an equation of induction current i_{LP}, where k is a proportional constant.

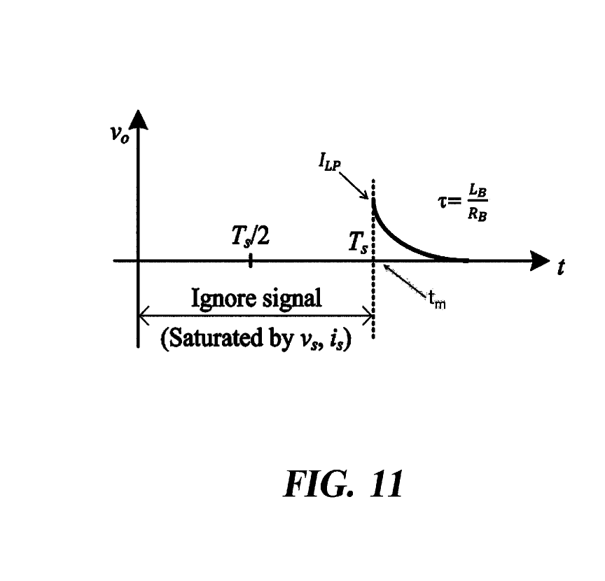

FIG. 11 is a diagram of an induced voltage detected by the receive coil 131.

Referring to FIGS. 8 to 11, an induction current is regenerated in the receive coil 131 by the induction current i_{LP} induced in the human body 810. The induced voltage of the receive coil 131 is then measured by the measuring unit 140. The measuring unit 140 does not measure induction current until T_{S}, but measures induction current T_{S}. Because it is common to use the receive coil 131 having a relatively small capacity, the magnetic flux is saturated by v_{S} and i_{S} until time T_{S}. Therefore, the measuring unit 140 measures the induced voltage of the receive coil 131 after time T_{S} at which the current application to the transmit coil 121 is stopped.

On the other hand, as shown in FIG. 9, the transmit pulse applied to the transmit coil 121 includes a positive voltage pulse (with the magnitude of V_{S}) and a negative voltage pulse (with the magnitude of −V_{S}), and the measuring unit 140 measures the induced voltage at a predetermined time after one positive voltage pulse (with the magnitude of V_{S}) and one negative voltage pulse are generated one after another.

For a predetermined time after time T_{S} at which the current application to the transmit coil 121 is stopped, a signal is not completely stopped at the transmit coil 121 due to the parasitic ringing phenomena. Therefore, in order to measure the induced voltage of the receive coil 131 after the parasitic ringing stops, the measuring unit 140 measures the induced voltage of the receive coil 131 at a time Tₘ after time T_{S}.

With the induction signal of the receive coil 131 measured at time Tₘ after time T_{S}, the whole or a part of the transmit coil 121 can double as the receive coil 131, obviating the need for separately providing the receive coil 131. In this case, the measuring unit 140 is connected in parallel with the transmit coil 121 that doubles as the receive coil, and the measuring unit 140 is designed to measure the induction signal after time T_{S} without measuring the induction signal until time T_{S}.

The positive voltage pulse and the negative voltage pulse of the transmit pulse to be applied to the transmit coil 121 can be formed to have the same pulse width, the same amplitude, and the same application time. Further, with different amplitudes thereof, the positive voltage pulse and the negative voltage pulse can be formed in a manner that a value obtained by multiplying the positive voltage pulse by its application time is equal to a value obtained by multiplying the negative voltage pulse and its application time.

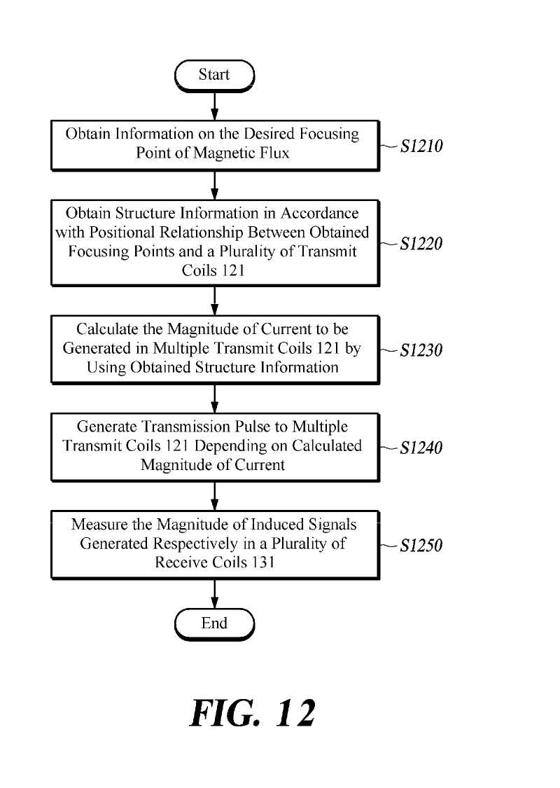

FIG. 12 is a flowchart of a method for high-resolution magnetic flux focusing according to some embodiments of the present disclosure.

The high-resolution magnetic-flux focusing method according to some embodiments of the present disclosure includes a step of acquiring information on a focusing point to focus a magnetic flux (Step S1210), a step of acquiring structure information based on the positional relationship between the focusing point and the transmit coils 121 (Step S1220), a step of calculating magnitudes of current to be generated and applied respectively to the plurality of transmit coils 121 based on the structure information (Step S1230), a step of generating transmit pulses to be applied to the plurality of transmit coils 121 based on the magnitudes of current (Step S1240), and a step of measuring an intensity of an induction signal generated at each of the receive coils 131 (Step S1250).

The present disclosure can be applied to various fields. For example, when focusing a magnetic flux on any one site of a human body where there is a cancer cell, heat is generated only at a small portion of the cancer cell, and hence it can be used as a means for destroying the cancer cell.

Further, in a hostage situation in a building, the apparatus according to some embodiments of the present disclosure may be used for transmitting a magnetic flux and measuring a current generated by a reflected magnetic flux to acquire information on the situation in the building through the wall, which can be used for military purposes.

In addition, the method and the apparatus according to some embodiments of the present disclosure can be applied to detect buried objects underground, such as underearth mineral resource.

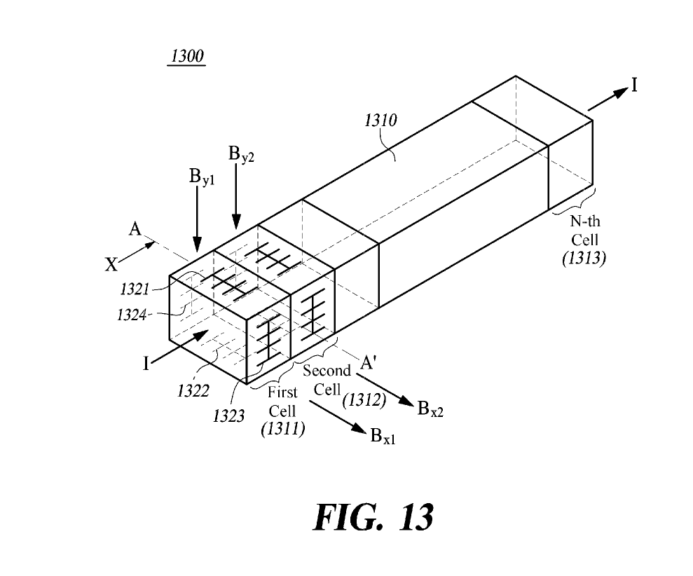

FIG. 13 is a perspective view of a magnetic-field receiving apparatus 1300 according to some embodiments of the present disclosure.

As shown in FIG. 13, the magnetic-field receiving apparatus 1300 according to some embodiments of the present disclosure includes a rod-like Hall element 1310 and a plurality of first pairs of electrodes 1321 and 1322 which are diametrically opposite each other with respect to the Hall element 1310 with reference to a first direction. For example, the opposing sides in the first direction indicate upper and lower surfaces facing each other in the Hall element 1310. The first pairs of electrodes 1321 and 1322 are arranged one after the other being spaced apart by a predetermined distance from each other.

The magnetic-field receiving apparatus 1300 further includes a plurality of second pairs of electrodes 1323 and 1324 which are diametrically opposite each other each other with respect to the Hall element 1310 in a second direction. The second direction is perpendicular to both the first direction and the longitudinal direction of the Hall element 1310. The second pairs of electrodes 1323 and 1324 are arranged one after the other being spaced apart by a predetermined distance from each other.

The Hall element 1310 is divided into n cells (a first cell 1311 to an n-th cell 1313) in the longitudinal direction, and each cell is arranged with at least one of the first pairs of electrodes 1321 and 1322 or the second pairs of electrodes 1323 and 1324.

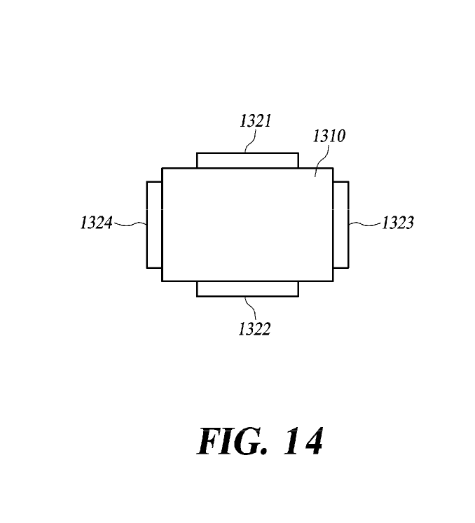

FIG. 14 is a cross-sectional view of the first cell 1311 in FIG. 13 taken along line A-A′ and viewed in direction X.

Referring to FIGS. 13 and 14, when a current having a predetermined magnitude is applied across the Hall element 1310 in the longitudinal direction and a magnetic flux is generated in a direction By1, a potential difference is generated at the second pair of electrodes 1323 and 1324 of the first cell 1311 depending on the magnetic flux intensity in direction By1. Likewise, with a magnetic flux generated in a direction Bx1, a potential difference is generated at the first pair of electrodes 1321 and 1322 of the first cell 1311 depending on the magnetic field intensity in direction Bx1. Similarly, when the magnetic flux is applied in directions Bx2 and By2, potential differences proportional to the intensities of Bx2 and By2 are induced at the first pair of electrodes and the second pair of electrodes of the second cell 1312.

That is, measuring the potential difference between the electrodes 1321 and 1322 allows the intensity of the magnetic field by direction Bx1 to be measured, and measuring the potential difference between the electrodes 1323 and the electrode 1324 allows the intensity of the magnetic field by direction By1 to be measured.

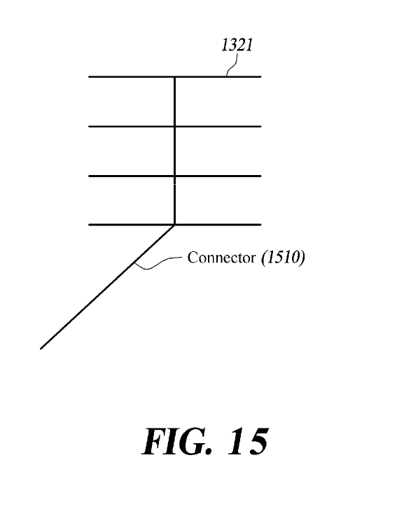

FIG. 15 is a diagram of the shape of an electrode that can be used in the magnetic-field receiving apparatus 1300.

As shown in FIG. 15, the electrodes 1321, 1322, 1323 and 1324 constituting the first pair of electrodes 1321, 1322 and the second pair of electrodes 1323, 1324 are in a wired configuration. That is, in order to maximally prevent the electrodes 1321, 1322, 1323 and 1324 from interrupting the flow of magnetic flux, the electrodes are formed by using a linear-shaped electrical conductor instead of forming them in a planar shape. Each of the electrodes 1321, 1322, 1323 and 1324 includes a connection line 1510 leading to the measuring unit 140. In FIG. 13, for simplicity of drawing, the connection line and the like are not shown.

The current flowing across the Hall element 1310 in the longitudinal direction of the Hall element 1310 is generated in the form of a pulse. In order to increase the accuracy of the measuring unit 140 with a single measurement of the magnetic flux, the potential difference can be measured at the pair of electrodes where the magnetic flux is generated by two consecutive generations of current pulse I.

Current I is applied with an appropriate magnitude. When the magnitude of current I is small, the dynamic range of the signal induced in each pair of electrodes is increased, but the SNR is decreased. On the other hand, the larger magnitude current I decreases the dynamic range of the signal induced in each pair of electrodes, but increases the SNR.

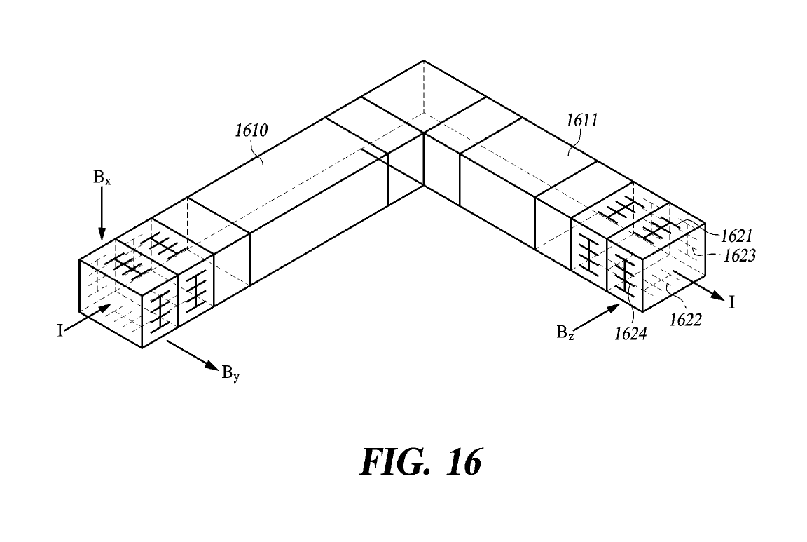

FIG. 16 is a diagram of a Hall element having two of the Hall element 1310 in FIG. 13 with one terminally protruding perpendicular from the other.

In the example shown in FIG. 13, it is hard for the magnetic-field receiving apparatus 1300 to detect magnetic field other than the magnetic flux in the directions of the x-axis and the y-axis, such as Bx and By. That is, it is hard to detect the magnetic flux in the direction of the z-axis (i.e., the longitudinal direction of the Hall element 1310).

Measuring the whole three-dimensional magnetic flux is provided by using a Hall element 1610 arranged in a planar manner as shown in FIG. 16.

The Hall element 1610 has a protrusion 1611 provided with electrodes, of which a first pair of electrodes 1621 and 1622 detects the magnetic flux in a direction Bx, and a second pair of electrodes 1623 and 1624 detects the magnetic flux in a direction Bz, and hence, the configuration shown in FIG. 16 enables to detect the magnetic flux components of all directions.

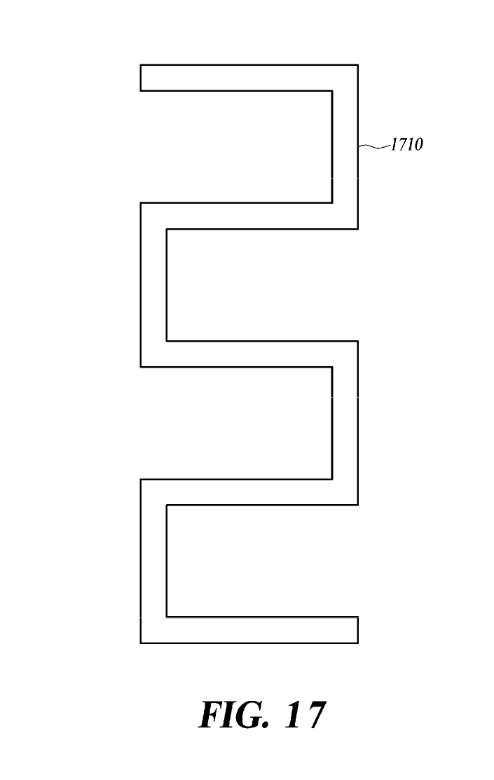

FIG. 17 is a diagram of the magnetic-field receiving apparatus 1300 with an optional zigzag configuration of the Hall element.

As shown in FIG. 17, arranging Hall elements 1710 in a planar zigzag form provides a planar zigzag pattern of the current flowing on the plane.

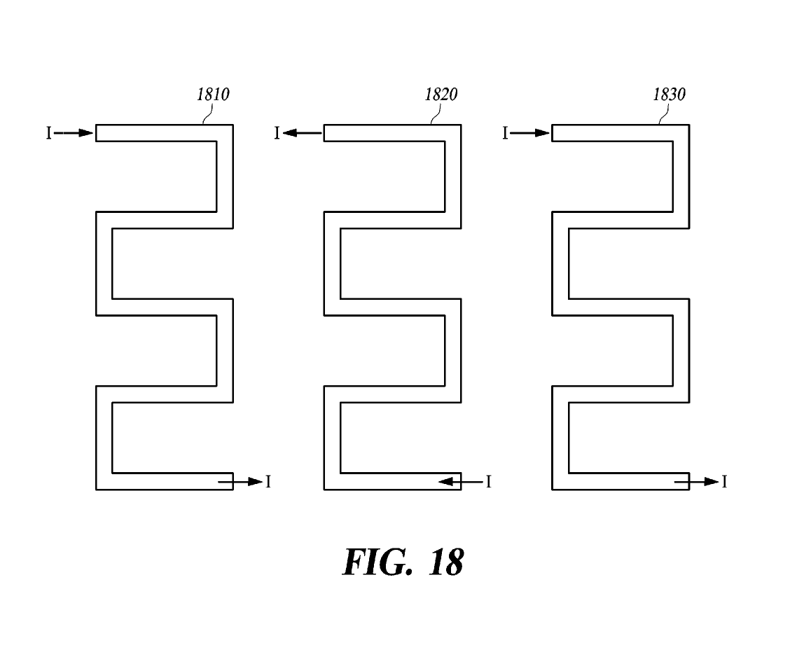

FIG. 18 is a diagram of a method for arranging a plurality of Hall elements.

As shown in FIG. 18, if Hall element current I were a pulsed current flowing across Hall elements 1810, 1820, and 1830, the Hall elements 1810, 1820 and 1830 are formed in the same shape and arranged in a row in order to cancel out the magnetic fields generated by the pulsed currents among the Hall elements 1810, 1820 and 1830. The Hall elements 1810, 1820 and 1830 are arranged so that the directions of currents between adjacent Hall elements 1810, 1820, and 1830 are opposite to each other. The Hall element is a magnetic sensor capable of measuring direction or intensity of a magnetic field by using the Hall effect.

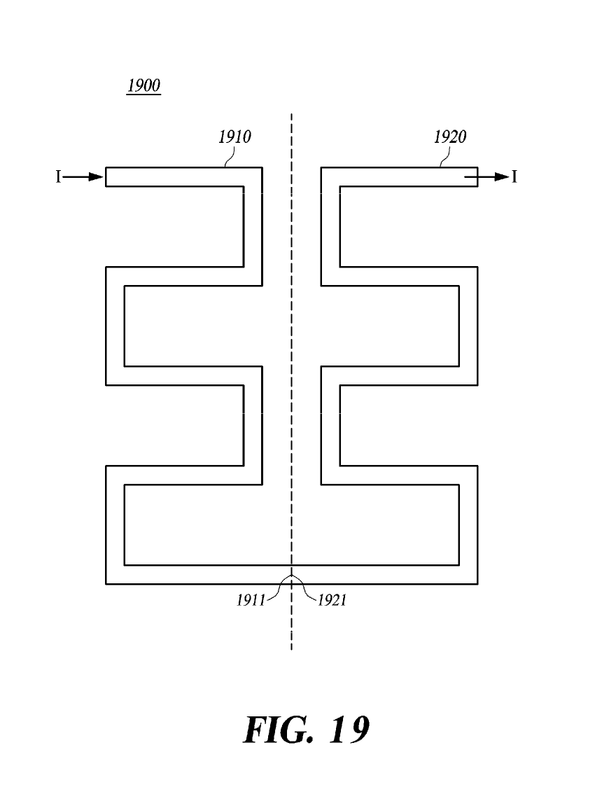

FIG. 19 is a diagram showing another example of the shape of the Hall element.

A Hall element 1900 shown in FIG. 19 has a zigzag shape similar to that of the Hall element shown in FIG. 18. In addition, the Hall element 1900 is composed of two adjacent Hall elements 1910 and 1920 that are arranged to face each other and interconnected at their terminals 1911 and 1921 on one side. In other words, one terminal 1911 of the first Hall element 1910 merges with one terminal 1921 of the second Hall element 1920. When the Hall element module 1900 includes the two Hall elements 1910 and 1920 conjoined as shown in FIG. 19, the magnetic fields between the two Hall elements 1910 and 1920 are canceled out each other.

Although exemplary embodiments of the present disclosure have been described for illustrative purposes, those skilled in the art will appreciate that various modifications, additions and substitutions are possible, without departing from the idea and scope of the claimed invention. Therefore, exemplary embodiments of the present disclosure have been described for the sake of brevity and clarity. Accordingly, one of ordinary skill would understand the scope of the claimed invention is not to be limited by the explicitly described above embodiments but by the claims and equivalents thereof.

### CROSS-REFERENCE TO RELATED APPLICATION

This application claims priority under 35 U.S.C §119(a) of Patent Application No. 10-2013-0127402, filed on Oct. 24, 2013 in Korea, the entire content of which is incorporated herein by reference. In addition, this non-provisional application claims priority in countries, other than the U.S., with the same reason based on the Korean patent application, the entire content of which is hereby incorporated by reference.

1. An apparatus for focusing a magnetic field, the apparatus comprising:
a magnetic-field generating unit including a plurality of transmit coils and configured to receive a transmit pulse to be applied to each of the transmit coils and to generate a magnetic field; and
a pulse generating unit configured to calculate a magnitude of a current to be applied to each of the transmit coils by using a structure information based on a positional relationship between a focusing point for focusing a magnetic flux and the plurality of transmit coils and to generate the transmit pulse to be applied to each of the plurality of transmit coils based on a calculated magnitude of the current.

2. The apparatus according to claim 1, further comprising:
a receive sensor unit including a plurality of receive coils each configured to generate an induction signal by a magnetic field focused on the focusing point; and
a measuring unit configured to measure a magnitude of the induction signal generated by each of the plurality of receive coils.

3. The apparatus according to claim 2, wherein the measuring unit is configured to measure the induction signal at a predetermined time after the transmit pulse is generated.

4. The apparatus according to claim 1, wherein the transmit pulse includes a positive-voltage pulse and a negative-voltage pulse.

5. The apparatus according to claim 1, wherein the pulse generating unit includes
a focusing-point calculating unit configured to calculate an information on the focusing point,
a structure-information calculating unit configured to calculate structure informations based on positional relationships of a plurality of focusing points with respect to a plurality of transmit coils, and
a circuit driving unit configured to generate the transmit pulse based on the information on the focusing point and the structure informations.

6. The apparatus according to claim 5, wherein the focusing-point calculating unit is configured to receive an information on one or more focusing points which are candidates for focusing point setting.

7. An apparatus for receiving a magnetic field, the apparatus comprising:
a Hall element having a rod shape; and
an electrode array including externally at least one of a plurality of first pairs of electrodes or a plurality of second pairs of electrodes, wherein
the first pairs of electrodes are diametrically opposite each other with respect to the Hall element with reference to a first direction,
the second pairs of electrodes are diametrically opposite each other with respect to the Hall element with reference to a second direction, and
the first direction is perpendicular to the second direction and a longitudinal direction of the Hall element.

8. The apparatus according to claim 7, wherein
the Hall element is divided into a plurality of cells in the longitudinal direction, and
each of the cells includes at least one of a first pair of electrodes or a second pair of electrodes.

9. The apparatus according to claim 8, wherein an electrode constituting the first pair of electrode and the second pair of electrode includes a linear-shaped electrical conductor.

10. The apparatus according to claim 8, wherein the Hall element is shaped to define a zigzag pattern of current flowing across the Hall element.

11. The apparatus according to claim 10, further comprising multiples of the Hall element in an array so that adjacent Hall elements have currents which are directed opposite to each other.

12. The apparatus according to claim 8, wherein
the Hall element is shaped to define a zigzag pattern of current flowing across the Hall element,
the apparatus has two of the Hall element configured so that two adjacent Hall elements are arranged to face each other and have terminals interconnected on one side.
